(19) United States
(12) Patent Application Publication
(10) Pub. No.: US 2022/0148179 A1
(43) Pub. Date: May 12, 2022

(54) METHOD FOR INSPECTING OPHTHALMIC DEVICE, INSPECTION JIG FOR OPHTHALMIC DEVICE, AND OPHTHALMIC DEVICE

(71) Applicant: KOWA COMPANY, LTD., Aichi (JP)

(72) Inventor: Nobuhiko TAKEUCHI, Tokyo (JP)

(21) Appl. No.: 17/429,298

(22) PCT Filed: Feb. 14, 2020
(86) PCT No.: PCT/JP2020/005697

§ 371 Date: Aug. 6, 2021

(30) Foreign Application Priority Data
Feb. 15, 2019 (JP) 2019-025957

(51) Int. Cl. G06T 7/00; G06T 7/90; G06T 7/11; A61B 3/00

(52) U.S. Cl. CPC G06T 7/0014; G06T 7/90; G06T 2207/30041; A61B 3/0008; G06T 7/11

(57) ABSTRACT

A method for inspecting an ophthalmic device which allows reflected light from an imaging inspection subject, which has been irradiated with light by using an illumination optical system of the ophthalmic device, to be received by using an image-capturing optical system of the ophthalmic device to capture an image of the imaging inspection subject; extracts color information from the captured image of the imaging inspection subject; and determines whether or not the color information satisfies a prescribed reference condition by comparing the extracted color information with reference information. An inspection jig for an ophthalmic device is used in the method for inspecting an ophthalmic device. Furthermore, the ophthalmic device includes the inspection jig.

### TECHNICAL FIELD

The present invention relates to a method for inspecting an ophthalmic device, an inspection jig for an ophthalmic device, and an ophthalmic device.

### BACKGROUND ART

The widespread use of portable information terminals such as smartphones and tablet terminals leads to an increase in the number of dry eye patients who have developed a condition so-called dry eye. Dry eye is an ophthalmic disease resulting from a part of the cornea surface being exposed to the outside without being protected by the tear fluid layer due to a reduction in the tear fluid layer covering the cornea surface. Developing dry eye may cause severe diseases such as corneal epitheliopathy and disorders of the conjunctiva, and in some cases, in complication of both. Thus dry eye needs to be properly examined and treated.

Examples of dry eye inspection devices include a tear fluid layer observation device (e.g., Patent Literature 1) that applies specific examination light to the subject eye and observes interference fringes on the tear fluid layer. This tear fluid layer observation device makes it possible to observe the state of the tear fluid surface of the cornea in a non-contact/non-invasive manner and thus possess the advantages of enabling examination to be performed easily, without imposing excessive physical burdens on the subject.

On the other hand, in the case of ophthalmic devices, such as the tear fluid layer observation device, where the device is equipped with an illumination optical system directed for the subject eye or an image-capturing optical system for imaging the irradiated subject eye, after the acquisition of the device, changes on the surface or the internal state of each optical element or the position/angle relation between the optical elements may occur, for example, due to the environment in which it is used or the like. In general, each optical element of the ophthalmic device is very precisely set up, and thus the state (quality) of the obtained image of the subject eye can be significantly changed by a slight change in the abovementioned matters. Thus, such an ophthalmic device is desirably provided, for example, with an inspection method of the optical elements described above which is possible to be performed on the user side.

In this regard, Patent Literature 2 discloses an example of an inspection (correction) technique of the ophthalmic device. The technique described in Patent Literature 2 includes a reflection plate that is disposed near the position of the focal point of the laser beam. The reflection plate is disposed in such a manner that the reflected light from the front surface or the reflected light from the back surface from the reflection plate caused by the laser beam is not received by a light receiving unit, and the correction is performed by measuring the scattered light from the reflection plate irradiated with the laser beam.

### CITATION LIST

### Patent Literature

Patent Literature 1: Japanese Patent Application Laid-Open No. Hei. 10-033483
Patent Literature 2: Japanese Patent Application Laid-Open No. 2007-082640

### SUMMARY OF INVENTION

### Technical Problem

However, in the technique disclosed in Patent Literature 2, the information of the scattered light intensity obtained from the inside of the reflection plate does not directly reflect information visually apparent in the image, such as shape features and color information of the reflection plate. Thus, the technique disclosed in Patent Literature 2 is not suitable for inspecting the image obtained by the tear fluid layer observation device described above.

Further, in the technique disclosed in Patent Literature 2, the positional relation and the like of each equipment part used for correction needs to be adjusted so as to receive only the scattered light from the inside of the reflection plate, but not the reflected light from the front surface and the back surface of the reflection plate. Thus, performing the correction requires a considerable amount of labor. Furthermore, the abovementioned reflection plate is a member functioning as a pseudo-anterior chamber of the subject eye, and thus the reflection plate needs to possess scattering characteristics similar to that of proteins inside the anterior chamber. As a result, the technique disclosed in Patent Literature 2 requires preparation of specific reflection plates with such characteristics, causing an increase in manufacturing costs accordingly.

With the aim of solving these problems, an object of the present invention is to provide a method for inspecting an ophthalmic device which enables the inspection of ophthalmic device that captures images of a subject eye, such as a tear fluid layer observation device, without imposing excessive labor burdens for adjusting each equipment part and which can perform quality determination of the image capturing capability of a device, determining whether or not a captured image accurately reflects the state of an imaging subject at high reproducibility. Furthermore, another object of the present invention is to provide an inspection jig used for performing the method for inspecting an ophthalmic device and an ophthalmic device including the inspection jig.

### Solution to Problem

For solving the abovementioned problems, the method for inspecting an ophthalmic device according to the present invention is characterized by including:

a step of allowing reflected light from an imaging inspection subject, which has been irradiated with light by using an illumination optical system of the ophthalmic device, to be received by using an image-capturing optical system of the ophthalmic device to capture an image of the imaging inspection subject;

a step of extracting color information from the captured image of the imaging inspection subject; and

a step of determining whether or not the color information satisfies a prescribed reference condition by comparing the extracted color information with reference information.

According to the abovementioned aspect of the present invention, the inspection is performed by using the reflected light from the imaging inspection subject without imposing excessive burdens for inspection work and preparatory work of the inspection. Furthermore, the inspection is performed on the basis of the color information directly showing the appearance state of the image of the imaging inspection subject captured during the inspection, making it possible to perform the quality determination of the image capturing capability of the ophthalmic device at high reproducibility. Note that the method for inspecting an ophthalmic device according to the present invention can be used not only for the tear fluid layer observation device, but also for other ophthalmic devices including the illumination optical systems and the image-capturing optical systems for inspection subjects, such as, for example, a fundus examination device.

Furthermore, in the method for inspecting an ophthalmic device according to the present invention, the reference condition preferably includes a condition that determines whether or not at least a hue value, among pieces of the extracted color information, is within a range of a hue value defined by the reference information.

According to the abovementioned aspect of the present invention, the quality determination of the image capturing capability of the ophthalmic device is performed on the basis of the hue value, among pieces of the color information of the image of the imaging inspection subject, which is capable of identifying a color difference more clearly. This can further improve accuracy of the determination.

Furthermore, in the method for inspecting an ophthalmic device according to the present invention, it is preferable that brightness information is extracted from the captured image of the imaging inspection subject and the extracted brightness information is compared with the reference information to determine whether or not the brightness information satisfies the prescribed reference condition.

According to the abovementioned aspect of the present invention, the inspection is performed further by using the brightness information of the image of the imaging inspection subject. This can effectively prevent the situation in which the reflected light quantity from the imaging inspection subject falls out of a prescribed acceptable range due to, for example, a trouble in a light source for illumination or the like and the state of the imaging inspection subject is not accurately captured. This can further improve accuracy of the determination.

Furthermore, in the method for inspecting an ophthalmic device according to the present invention, it is preferable that the imaging inspection subject is disposed in a position opposed to an objective lens-barrel of the ophthalmic device where the subject eye is placed during the examination to capture the image of the imaging inspection subject.

According to the abovementioned aspect of the present invention, the position of the imaging inspection subject is matched with the position of the subject eye during the examination, making it possible to capture the image of the imaging inspection subject in a state of closing to that of the subject eye during the examination. This can further improve accuracy of the determination.

Furthermore, in the method for inspecting an ophthalmic device according to the present invention, it is preferable that an inspection area is cut out from the captured image and at least the color information is extracted from the inspection area thus cut out.

According to the abovementioned aspect of the present invention, cutting out the inspection area from the image of the imaging inspection subject makes it possible to perform accurate comparison with the reference information. This can further improve accuracy of the determination.

Furthermore, the inspection jig for an ophthalmic device according to the present invention is characterized by being used in the method for inspecting an ophthalmic device and including the imaging inspection subject.

According to the abovementioned aspect of the present invention, the inspection is performed by using the reflected light from the imaging inspection subject without imposing excessive burdens for inspection work and preparatory work of the inspection. Furthermore, the color information directly showing the reflection characteristics of the imaging inspection subject captured during the inspection can be provided from the reflected light of the imaging inspection subject, making it possible to perform the quality determination of the image capturing capability of the ophthalmic device at high reproducibility.

Furthermore, in the inspection jig for an ophthalmic device according to the present invention, an optical thin film that reflects the illumination light from the illumination optical system is preferably formed at least on the front surface of the imaging inspection subject.

According to the abovementioned aspect of the present invention, the optical thin film is provided at least on the front surface, and thus the reflected light including the color information can be more clearly obtained. This makes it possible to, for example, more clearly identify a difference in the color information between the image of the imaging inspection subject captured during the inspection and the reference image for inspection. As a result, accuracy of the determination can be further improved.

Furthermore, in the inspection jig for an ophthalmic device according to the present invention, the optical thin film preferably causes specular reflection of the illumination light.

According to the abovementioned aspect of the present invention, the reflected light of a specific wavelength band can be selectively obtained from the optical thin film, making it possible to, for example, facilitate the comparison with the reference information.

Furthermore, in the inspection jig for an ophthalmic device according to the present invention, the optical thin film preferably includes a plurality of portions with different thicknesses.

According to the abovementioned aspect of the present invention, the reflected light of a plurality of specific wavelength bands can be obtained from the optical thin film, making it possible to increase selectable options of the color information at the time of the comparison with the reference information.

Furthermore, in the inspection jig for an ophthalmic device according to the present invention, it is preferable that the inspection jig further includes a retaining portion for the imaging inspection subject that retains the imaging inspection subject and the retaining portion for the imaging inspection subject is attached to a tip of the objective lens-barrel.

According to the abovementioned aspect of the present invention, the position of the imaging inspection subject is matched with the position of the subject eye during the examination, making it possible to capture the image of the imaging inspection subject in a state of closing to that of the subject eye during the examination. This can further improve accuracy of the determination.

Furthermore, the ophthalmic device according to the present invention is characterized by including the inspection jig of an ophthalmic device.

According to the abovementioned aspect of the present invention, the inspection is performed by using the reflected light from the imaging inspection subject without imposing excessive burdens for inspection work and preparatory work of the inspection. Furthermore, the color information directly showing the reflection characteristics of the imaging inspection subject captured during the inspection can be provided from the reflected light of the imaging inspection subject, making it possible to perform the quality determination of the image capturing capability of the ophthalmic device at high reproducibility.

### Advantageous Effects of Invention

According to the present invention, there can be provided the method for inspecting an ophthalmic device which enables the inspection of the ophthalmic device that captures images of a subject eye, such as a tear fluid layer observation device, without imposing excessive labor burdens for adjusting each equipment part, and which can perform quality determination of the image capturing capability of the ophthalmic device at high reproducibility because the captured image is a reflection image that accurately reflects the reflection characteristics of the imaging subject. Further, the inspection jig used for performing the method for inspecting an ophthalmic device and the ophthalmic device including the inspection jig can be provided.

### BRIEF DESCRIPTION OF DRAWINGS

FIG. 1 is a schematic view of an ophthalmic device according to the present embodiment.

FIG. 2 is a flowchart showing a procedure of a method for inspecting an ophthalmic device according to the present embodiment.

FIG. 3 is a diagram describing a procedure for cutting out an inspection area from a captured image of an imaging inspection subject according to the present embodiment.

FIG. 4 is another diagram describing the procedure for cutting out the inspection area from the captured image of the imaging inspection subject according to the present embodiment.

FIG. 5 is yet another diagram describing the procedure for cutting out the inspection area from the captured image of the imaging inspection subject according to the present embodiment.

FIG. 6 is a flowchart showing the procedure for cutting out the inspection area from the captured image of the imaging inspection subject according to the present embodiment.

FIG. 7 is an exploded perspective view of an inspection jig of the ophthalmic device according to the present embodiment.

FIG. 8 is a vertical cross-sectional view of the inspection jig of the ophthalmic device according to the present embodiment attached to a tip of an objective lens-barrel.

FIG. 9 is a vertical cross-sectional view of the imaging inspection subject according to the present embodiment.

### DESCRIPTION OF EMBODIMENTS

Hereinafter, an ophthalmic device, a method for inspecting an ophthalmic device, and an inspection jig of an ophthalmic device according to an embodiment of the present invention will be described in detail with reference to the drawings.

<Ophthalmic Device>

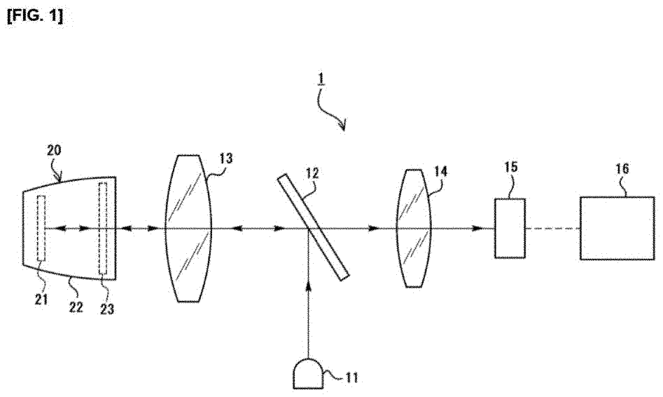

First, a schematic configuration of an ophthalmic device 1 according to the present embodiment will be described with reference to FIG. 1. FIG. 1 is a schematic view of the ophthalmic device 1. As shown in FIG. 1, the ophthalmic device 1 includes a light source 11 (a white light source in the present embodiment), a half mirror 12, an objective lens 13, an image-forming lens 14, an image-capturing unit 15, an analysis device 16, an inspection jig 20, and the like. Note that the ophthalmic device 1 according to the present embodiment is a tear fluid layer observation device. However, the ophthalmic device 1 is not limited to the tear fluid layer observation device and, for example, it may be other ophthalmic devices such as a fundus examination device.

Light from the light source 11 is irradiated toward the half mirror 12 and a part of the light having reached the half mirror 12 is reflected toward the objective lens 13. Further, the light having passed through the objective lens 13 reaches an imaging inspection subject 21 installed in the inspection jig 20. In this manner, the imaging inspection subject 21 is irradiated. In the actual ophthalmic examination, a subject eye is placed in the position where the imaging inspection subject 21 is disposed and the subject eye is irradiated with the illumination light having passed through the objective lens 13.

Further, when the illumination light is irradiated to the imaging inspection subject 21 of the inspection jig 20, the illumination light is reflected from the imaging inspection subject 21 toward the objective lens 13. The reflected light passes through the objective lens 13 and the half mirror 12 and is converged by the image-forming lens 14. Finally, the converged reflected light is received by the image-capturing unit 15 (e.g., a CCD camera, a CMOS camera, etc.) and converted into electric signals that form image information of the imaging inspection subject 21. In this manner, an image of the imaging inspection subject 21 is captured.

Further, the image information of the imaging inspection subject 21 captured by the image-capturing unit 15 is sent to the analysis device 16. The analysis device 16 described herein includes a processing unit that processes data and arithmetic operations and a storage unit that stores data. The storage unit stores, for example, a program for executing the image processing described below and prescribed data and the processing unit performs data processing in conformity to prescribed commands from the program and the like. Without being particularly limited thereto, the analysis device 16 may be a common computer which includes, for example, a CPU, a RAM, a hard disk, input and output devices, and a communication unit.

Note that, in the following description, a set of optical elements for illuminating the imaging inspection subject 21 disposed in an optical path from the light source 11 to the imaging inspection subject 21 is referred to as an illumination optical system. That is, in the aspect shown in FIG. 1, the light source 11, the half mirror 12, and the objective lens 13 are included in the illumination optical system. Other optical elements such as various lenses, mirrors, and filters may be included in the illumination optical system as needed.

Further, a set of optical elements for capturing the image of the imaging inspection subject 21 disposed in an optical path from the imaging inspection subject 21 to the image-capturing unit 15 is referred to as an image-capturing optical system. In the aspect shown in FIG. 1, the objective lens 13, the half mirror 12, the image-forming lens 14, and the image-capturing unit 15 are included in the image-capturing optical system. Other optical elements such as various lenses, mirrors, and filters may be included in the image-capturing optical system as needed.

### <Inspection Method of Ophthalmic Device>

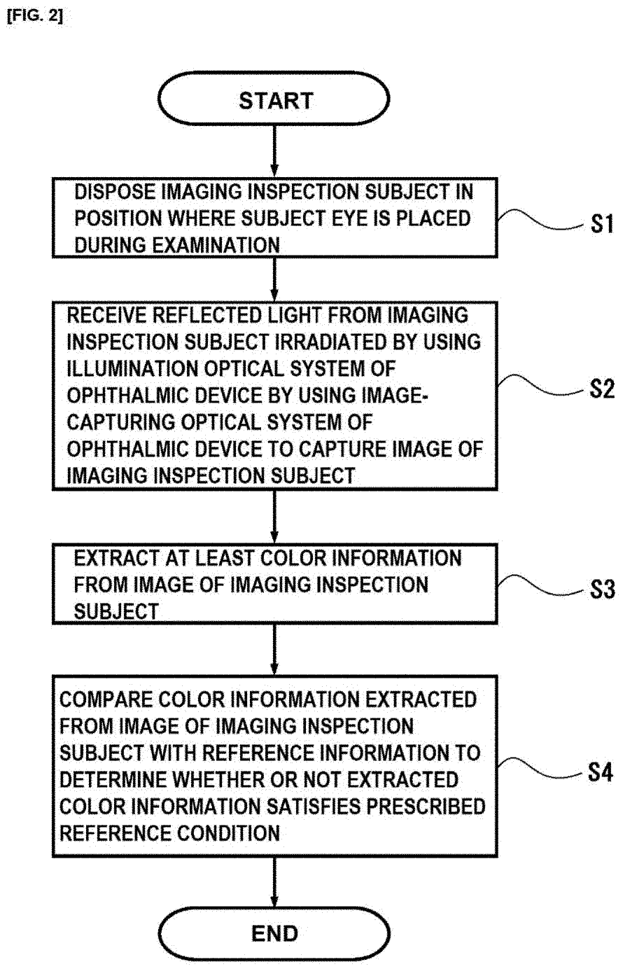

Next, the abovementioned method for inspecting the ophthalmic device 1 will be described with reference to FIG. 2. FIG. 2 is a flowchart showing a procedure of the method for inspecting the ophthalmic device 1 according to the present embodiment. First, the imaging inspection subject 21 is placed in the position where the subject eye is placed during the examination (e.g., the position opposed to an objective lens-barrel of the ophthalmic device 1) (S1).

The imaging inspection subject 21 in the present embodiment retains the state of being housed inside a retaining portion 22, for an imaging inspection subject, of the inspection jig 20. For example, the imaging inspection subject 21 is placed in the position where the subject eye is placed during the examination by attaching the retaining portion 22 for an imaging inspection subject to a tip 31 of an objective lens-barrel 30 (shown in FIG. 8). Note that the position of the imaging inspection subject 21 is not limited thereto.

Next, the reflected light from the imaging inspection subject 21, which has been irradiated with light by using the illumination optical system of the ophthalmic device 1, is received by using the image-capturing optical system of the ophthalmic device 1 to capture the image of the imaging inspection subject 21 (S2). More specifically, as described above, the imaging inspection subject 21 is irradiated with light from the light source 11 and the reflected light from the imaging inspection subject 21, thus irradiated, is received by the image-capturing unit 15. In this manner, the image of the imaging inspection subject 21 is captured and recorded in the image-capturing unit 15.

Next, the image recorded in the image-capturing unit 15 is sent to the analysis device 16 where at least the color information is extracted from the image of the imaging inspection subject 21 by image processing executable by the analysis device 16 (S3). More preferably, a hue value is extracted from the image of the imaging inspection subject 21.

Note that, before extracting the color information from the image of the imaging inspection subject 21, an inspection area may be cutout in advance from the captured image and the color information may be extracted from the inspection area thus cut out. A selection method of the inspection area to be cut out is not limited to particular ones. However, examples of the selection method include a method in which, an image area (inspection area) having the luminance greater than or equal to a predetermined threshold value and having little color unevenness is selected by the image processing of the analysis device 16 by referring to a luminance value and an RGB value of each pixel of the image of the imaging inspection subject 21. Note that the inspection area thus cut out is hereinafter referred to as “inspection area S”.

An example of the method for cutting out the inspection area S will be specifically described below (note that the method for cutting out the inspection area S is not limited to the following). The method for cutting out the inspection area S in the present embodiment include the following Step 1 to Step 4.

(1) Step 1: determining the length of the inspection area S
(2) Step 2: determining the width of the inspection area S
(3) Step 3: cutting out the captured image in the inspection area S
(4) Step 4: calculating a hue value and brightness of HSV values of the cut out captured image

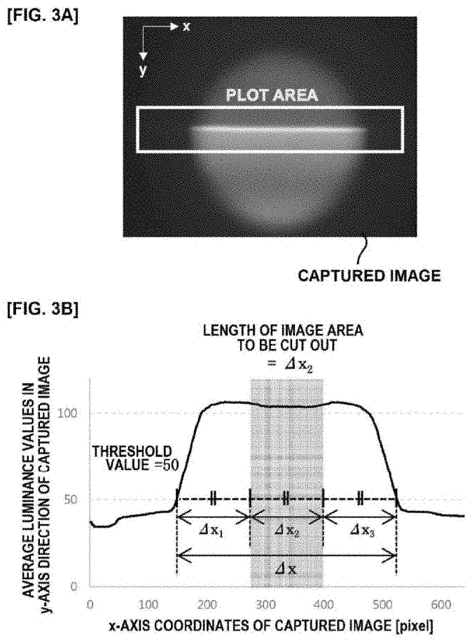

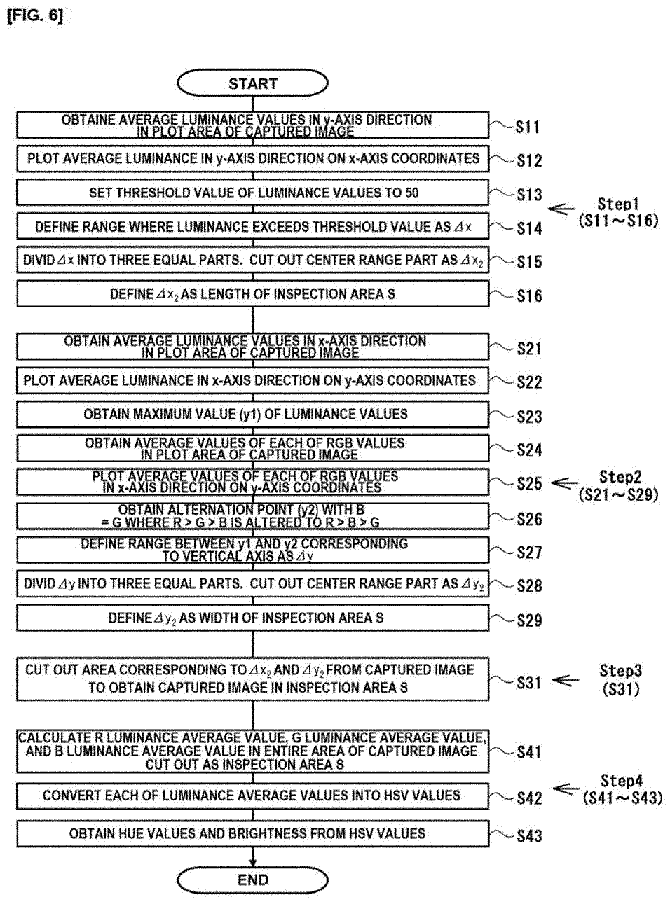

With reference to FIG. 3 to FIG. 5 and a flowchart in FIG. 6, each of the abovementioned Step 1 to Step 4 will be described in detail. As described above, Step 1 is a step for determining the length of the inspection area S. First, the average luminance values in the y-axis direction in the plot area shown in FIG. 3(a) are obtained (S11 in FIG. 6). The average luminance values are plotted on the x-axis coordinates (S12 in FIG. 6) as shown in the graph in FIG. 3(b). The horizontal axis of this graph corresponds to the x-axis coordinates of the captured image. Furthermore, values of the vertical axis of this graph correspond to the abovementioned average luminance values in the y-axis direction. More specifically, the values of the vertical axis of the graph are obtained by adding the luminance values of the group of pixels along the y axis which possess the same x-axis coordinates and averaging these added values (e.g., a value obtained by adding the luminance value of the pixel P11 (x=a0, y=b1), the luminance value of the pixel P12 (x=a0, y=b2), the luminance value of the pixel P13 (x=a0, y=b3), . . . , the luminance value of the pixel P1n (x=a0, y=bn) in the plot area and averaging the total value). Next, a threshold value (set to 50) is set to the vertical axis (S13 in FIG. 6). A range where the luminance exceeds the threshold value is defined as Δx (S14 in FIG. 6). This Δx is divided into three equal parts and the center range part is cut out as Δx2 (S15 in FIG. 6). This Δx2 corresponds to the length of the inspection area S (S16 in FIG. 6).

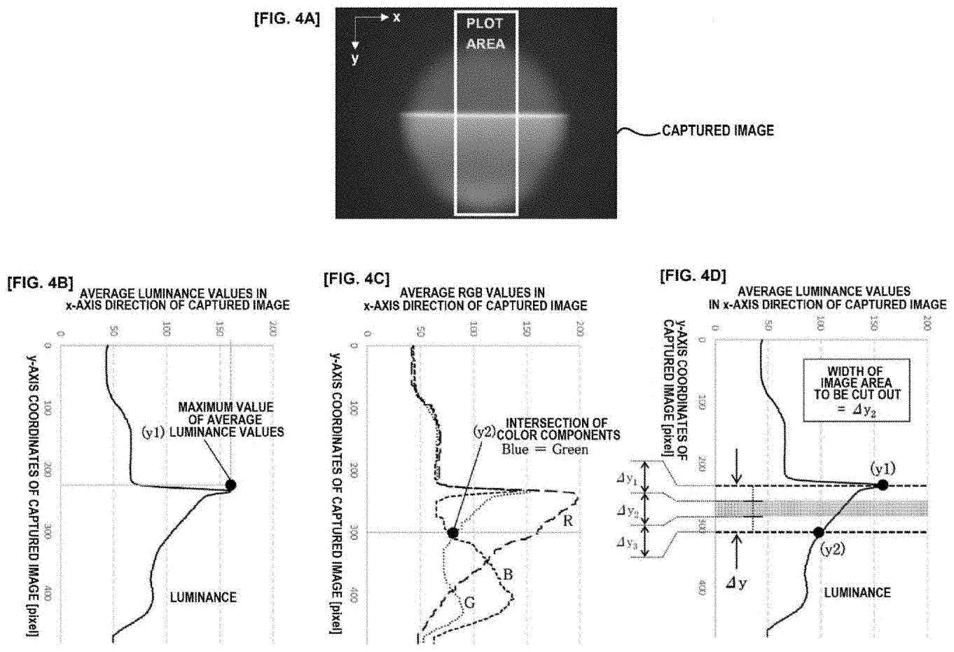

Next, Step 2 is a step for determining the width of the inspection area S. First, the average luminance values in the x-axis direction in the plot area shown in FIG. 4(a) are obtained (S21 in FIG. 6). The average luminance values are plotted on the y-axis coordinates (S22 in FIG. 6) as shown in the graph in FIG. 4 (b). The vertical axis of this graph corresponds to the y-axis coordinates of the image. Furthermore, values of the horizontal axis of this graph correspond to the abovementioned average luminance values in the x-axis direction. More specifically, the values of the horizontal axis of the graph are obtained by adding the luminance values of the group of pixels along the x axis which possess the same y-axis coordinates and averaging these added values (e.g., a value obtained by adding the luminance value of the pixel P21 (x=a1, y=b0), the luminance value of the pixel P22 (x=a2, y=b0), the luminance value of the pixel P23 (x=a3, y=b0), . . . , the luminance value of the pixel P2n (x=an, y=b0) in the plot area and averaging the total value). Next, the maximum value of the luminance values is obtained (S23 in FIG. 6). In FIG. 4(b), y1 corresponds to the maximum value of the luminance. Next, the average value of each RGB in the x-axis direction in the plot area is obtained (S24 in FIG. 6). These average values are plotted on the y-axis coordinates (S25 in FIG. 6) as shown in the graph in FIG. 4 (c). The horizontal axis of the graph shown in FIG. 4 (c) corresponds to the R luminance, the G luminance, and the B luminance averaged in the x-axis direction. The vertical axis of the graph corresponds to the y-axis coordinates of the image. Next, attention is focused on each profile of the R luminance, the G luminance, and the B luminance. Using the profiles, an alteration point where the relation of each luminance value of RGB is altered from R>G>B to R>B>G is obtained (S26 in FIG. 6). In FIG. 4 (c), y2 corresponds to the abovementioned alteration point. Next, a range between y1 and y2 corresponding to the vertical axis is defined as Δy (S27 in FIG. 6). This Δy is divided into three equal parts and the center range part is cut out as Δy2 (S28 in FIG. 6). As shown in FIG. 4 (d), this Δy2 corresponds to the width of the inspection area S (S29 in FIG. 6).

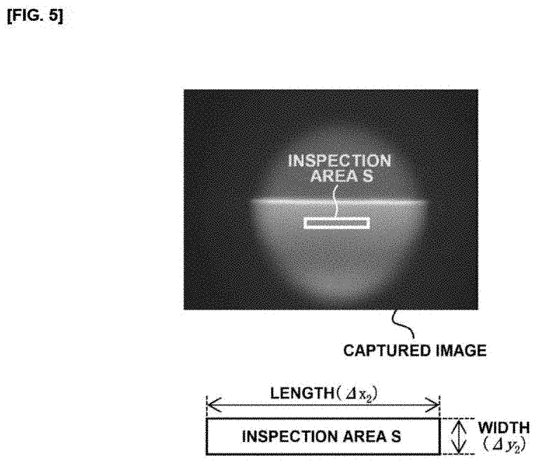

Next, Step 3 is a step for cutting out the range corresponding to the inspection area S defined as described above from the captured image. As shown in FIG. 5, the area corresponding to Δx2 selected in Step 1 and Δy2 selected in Step 2 is cut out from the captured image (S31 in FIG. 6). The cut-out image is the captured image in the inspection area S.

Next, Step 4 is a step for calculating a hue value and brightness of HSV values of the captured image cut out as the inspection area S. The R luminance average value, the G luminance average value, and the B luminance average value in the entire area of the captured image cut out as the inspection area S are calculated (S41 in FIG. 6). These values are converted into the HSV values (S42 in FIG. 6). The hue value and brightness are acquired from these HSV values (S43 in FIG. 6).

Note that conversion from the RGB values to the HSV values can be performed by using a known conversion formula shown in, for example, Japanese Patent Application Laid-Open No. 2019-009752. Specifically, the inputted color information of each pixel is converted from the RGB values to the HSV (hue, saturation, brightness) values by a conversion program stored in the analysis device 16. The color information may further include a color difference value. However, the conversion values from the RGB values are not limited to HSV, and the conversion values may be values belonging to other color models such as HSB and HSL.

With reference to FIG. 2 again, the subsequent procedure of the inspection method according to the present embodiment will be described. After the abovementioned procedure of S3 shown in FIG. 2, the color information extracted from the image of the imaging inspection subject 21 is compared with the reference information to determine whether or not the extracted color information satisfies a prescribed reference condition (S4). In the present embodiment, this procedure according to S4 is also processed by the analysis device 16. In this process, the reference information in the present embodiment corresponds to the color information of the image of the imaging inspection subject 21 captured by using the ophthalmic device 1 at the pre-shipment stage of the ophthalmic device 1 (hereinafter “reference image”). However, the reference information is not limited to the reference image as long as the information can be compared with the color information or the like extracted from the image of the imaging inspection subject 21.

By comparison with the color information of the reference image, it is determined whether or not the hue value, for example, of the inspection area S of the inspection subject falls in a prescribed acceptable range. In this process, as an example of setting the acceptable range, the acceptable range is set at a numerical value range of which, using the hue value of a comparison image area C in the reference image corresponding to the inspection area S as the median, the lower limit is defined by a value obtained by subtracting the prescribed number from the median and the upper limit is defined by a value obtained by adding the prescribed number to the median. In this case, for example, where the hue value of the comparison image area C is 19° and the acceptable range is set to ±30% (in the case where the lower limit is set to 13.3° and the upper limit is set to 24.7° in the acceptable range), if the hue value of the inspection area S is 15°, it is determined that the color information extracted from the image of the imaging inspection subject 21 satisfies the reference condition. As a result, it can be confirmed that the image captured by using the ophthalmic device 1 accurately reflect the state of the imaging subject. This shows that the ophthalmic device 1 has the excellent image capturing capability.

According to the present embodiment, the image captured by using the ophthalmic device 1 is confirmed whether or not it accurately reflects the state of the imaging subject on the basis of the color information (in particular, the hue value) directly showing the state of the image of the imaging inspection subject 21, making it possible to perform the quality determination of the image capturing capability of the ophthalmic device 1 at high reproducibility.

Furthermore, according to the present embodiment, only a simple procedure of attaching the inspection jig 20 to the tip 31 of the objective lens-barrel 30 of the ophthalmic device 1 (shown in FIG. 8) is necessary to place the imaging inspection subject 21 in the prescribed inspection position and capture the image of the imaging inspection subject 21. Thus, even a user who possesses no special skills in the inspection can easily perform each of the inspection procedures described above.

Thus far, a description has been provided regarding the aspect in which the color information (the hue value) extracted from the image of the imaging inspection subject 21 is used as the information to be compared with the reference information. However, the brightness information (brightness) may be used in addition to the color information.

Using the brightness information in addition to the color information can effectively prevent the situation where the state of the imaging inspection subject 21 cannot be accurately captured such as in the case where, for example, that the light quantity of the reflected light to be received by the image-capturing unit 15 exceeds the capacity of the light receiving quantity of the image-capturing unit 15 and becomes saturated. Furthermore, it can effectively prevent the situation where the state of the imaging inspection subject 21 cannot be accurately captured due to, for example, a disturbing light component, other than the reflected light from the imaging inspection subject 21, entering the image-capturing unit 15 and brightness of the image becoming significantly high. Note that a prescribed light control unit capable of adjusting the light quantity of the light source 11 or the light receiving quantity of the image-capturing unit 15 may be separately provided.

<Inspection Jig>

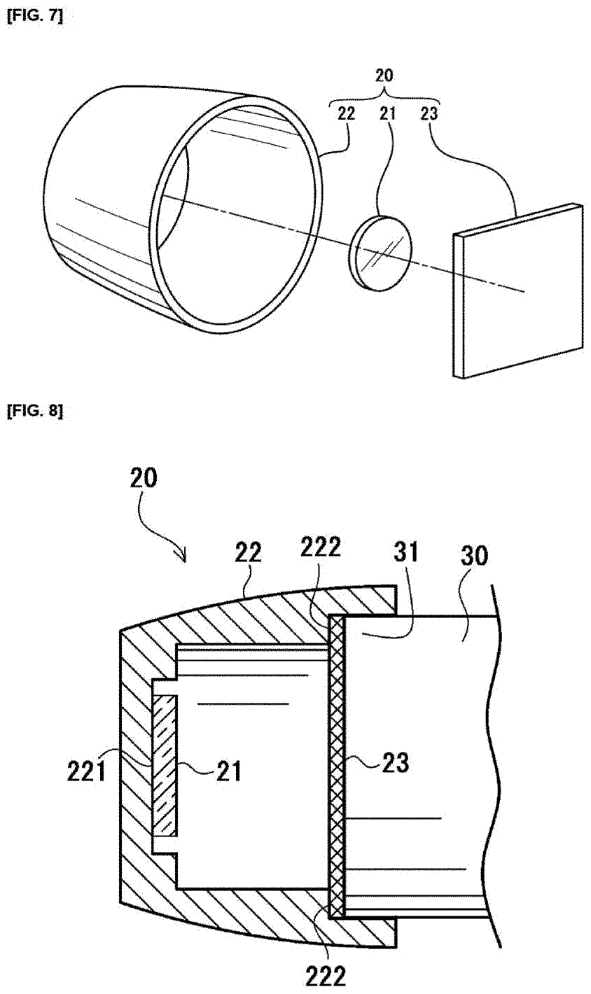

Next, with reference to FIG. 7 and FIG. 8, the inspection jig 20 according to the present embodiment will be described. FIG. 7 is an exploded perspective view of the inspection jig 20. FIG. 8 is a vertical cross-sectional view of the inspection jig 20 attached to the tip 31 of an objective lens-barrel 30.

As shown in FIG. 7, the inspection jig 20 includes the imaging inspection subject 21 and the retaining portion 22 for an imaging inspection subject described above. Further, the inspection jig 20 preferably includes an ND filter 23 that reduces the reflected light quantity from the imaging inspection subject 21. The imaging inspection subject 21 and the ND filter 23 are housed in the retaining portion 22 for an imaging inspection subject.

The retaining portion 22 for an imaging inspection subject in the present embodiment is a black bottomed cylindrical body with an opening top. If the disturbance light such as indoor light enters the imaging inspection subject 21, the original RGB values derived from the reflected light from the imaging inspection subject 21 cannot be obtained. In such a case, the reproducibility of the inspection may be impaired by an influence of the surrounding environment. Regarding this point, the provision of the abovementioned retaining portion 22 for an imaging inspection subject can prevent the disturbance light from entering the imaging inspection subject 21. In this manner, the original RGB values derived from the reflected light from the imaging inspection subject 21 can be reliably obtained without being influenced by the surrounding environment, making it possible to improve the reproducibility of the inspection.

Further, as shown in FIG. 8, a placing surface 221 where the imaging inspection subject 21 is placed is formed inside the retaining portion 22 for an imaging inspection subject. Without being limited to particular one, the imaging inspection subject 21 is retained to the placing surface 221 via an adhesive unit such as, for example, a double-sided adhesive tape.

Further, a supporting stage 222 that supports the ND filter 23 is formed inside the retaining portion 22 for an imaging inspection subject. Without being limited to particular one, a side edge of the ND filter 23 is retained to the supporting stage 222 via an adhesive unit such as, for example, a double-sided adhesive tape. Note that the ND filter 23 is preferably diagonally housed in the retaining portion 22 for an imaging inspection subject in order to prevent that, when the reflected light from the imaging inspection subject 21 enters the ND filter 23, a part of the incident light is re-reflected toward the imaging inspection subject 21.

In this configuration, the tip 31 of the objective lens-barrel of the ophthalmic device 1 is detachably fitted in the top opening of the retaining portion 22 for an imaging inspection subject, which retains the imaging inspection subject 21 and the ND filter 23, so that the inspection jig 20 is attached to the objective lens-barrel 30 of the ophthalmic device 1.

Note that the retaining portion 22 for an imaging inspection subject does not need to be the black bottomed cylindrical body described above. However, if the retaining portion 22 for an imaging inspection subject has a shape which cannot completely block the penetration of the disturbance light, other countermeasures such as capturing the image of the imaging inspection subject 21 in a dark environment are required.

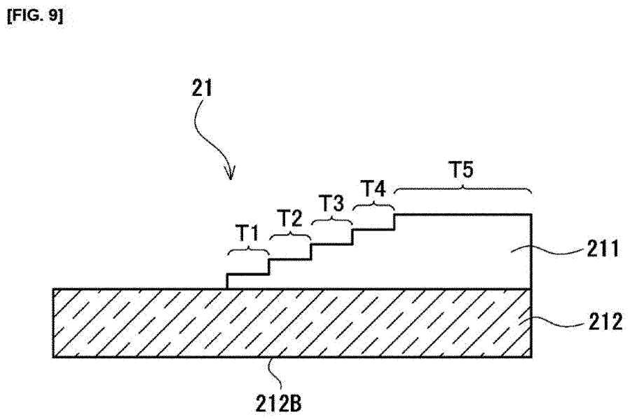

Next, with reference to FIG. 9, the imaging inspection subject 21 according to the present embodiment will be described. FIG. 9 is a vertical cross-sectional view of the imaging inspection subject 21. As shown in FIG. 9, the imaging inspection subject 21 includes an optical thin film 211 formed on the front surface side and a substrate 212.

The optical thin film 211 in the present embodiment is a vapor deposition film having a thickness of about 100 nm to 200 nm, without being limited to particular one. When the illumination light is irradiated on the optical thin film 211, the illumination light is reflected, for example, on the front surface and the back surface of the optical thin film 211. As a result, the reflected light from each surface interferes with each other and the reflected light of a specific wavelength band is outputted from the optical thin film 211. The wavelength band of the reflected light is preferably adjusted to, for example, a color corresponding or approximate to the coloration of the interference fringe of the subject eye obtained by using the tear fluid layer observation device by appropriately selecting the thickness of the optical thin film 211 and a material of the optical thin film 211 having a desired refractive index.

The material of the optical thin film 211 is not limited to particular one. Examples of the material may include titanium dioxide (TiO₂), zirconium dioxide (ZrO₂), and silicon dioxide (SiO₂). These materials, used for lens coatings, optical filters, and the like, are preferable, as the y are chemically stable, less prone to change in properties with time, inexpensive, and exhibits a desired interference color at high reproducibility. Further, a film forming method of the optical thin film 211 is not limited to particular one, and a film forming method belonging to conventionally used physical vapor deposition methods, a film forming method belonging to conventionally used chemical vapor deposition methods, and the like may be used.

Further, the optical thin film 211 in the present embodiment preferably includes a plurality of portions with different thicknesses, for example, those shown by reference signs T1 to T5 in FIG. 9. Disposing the plurality of portions with different thicknesses makes it possible to obtain the reflected light of different colors (wavelength bands) from each of the thickness portions. This can increase selectable options of the color information at the time of the comparison with the reference information. Note that, in FIG. 9, each of the thickness portions is arranged so as to increase the thickness from the left to the right of the drawing, thereby forming a stair shaped surface layer portion. However, a mode of the arrangement is not limited thereto.

The optical thin film 211 according to the present embodiment causes the specular reflection of the illumination light as described above. However, a mode of the reflection of the illumination light is not limited thereto. For example, the optical thin film 211 may be light scattering bodies such as various coating materials and ink, and the mode of the reflection may be a mode of causing scattering reflection of the illumination light. In a case of using the light scattering bodies such as various coating materials and ink, it is also desirable to select a material which is chemically stable and less prone to change in properties with time as described above. Note that various coating materials, ink, and the like are generally prone to change in properties with time. In a case of using such materials prone to change in properties with time, the reproducibility of the inspection can be maintained by regularly replacing the imaging inspection subject 21 with new one.

The substrate 212 in the present embodiment is a specular object made of glass suitable for forming the optical thin film, without being limited thereto. Note that, in order to prevent the situation where the illumination light is reflected on a back surface 212B of the substrate 212 and interrupts imaging of the optical thin film 211 (the imaging inspection subject 21), the back surface 212B is preferably subjected to frosting processing or black coating.

The embodiments of the present invention have been described in detail above. It should be understood that the abovementioned description is merely for facilitating the understanding of the present invention and not intended for limiting the present invention. The present invention can be modified or improved without departing from the gist of the invention. Further, the present invention includes its equivalents.

### REFERENCE SIGNS LIST

1 ophthalmic device
11 light source
12 half mirror
13 objective lens
14 image-forming lens
15 image-capturing unit
16 analysis device
20 inspection jig for ophthalmic device
21 imaging inspection subject
211 optical thin film
212 substrate
22 retaining portion for imaging inspection subject
23 ND filter
30 objective lens-barrel
31 tip of objective lens-barrel

1. A method for inspecting an ophthalmic device comprising:
a step of allowing reflected light from an imaging inspection subject, which has been irradiated with light by using an illumination optical system of the ophthalmic device, to be received by using an image-capturing optical system of the ophthalmic device to capture an image of the imaging inspection subject;
a step of extracting color information from the captured image of the imaging inspection subject; and
a step of determining whether or not the color information satisfies a prescribed reference condition by comparing the extracted color information with reference information.

2. The method for inspecting an ophthalmic device according to claim 1, wherein the reference condition includes a condition that determines whether or not at least a hue value, among pieces of the extracted color information, is within a range of a hue value defined by the reference information.

3. The method for inspecting an ophthalmic device according to claim 1, comprising: further extracting brightness information from the captured image of the imaging inspection subject; and comparing the extracted brightness information with the reference information to determine whether or not the brightness information satisfies the prescribed reference condition.

4. The method for inspecting an ophthalmic device according to claim 1, wherein the imaging inspection subject is disposed in a position opposed to an objective lens-barrel of the ophthalmic device where a subject eye is placed during examination to capture the image of the imaging inspection subject.

5. The method for inspecting an ophthalmic device according to claim 1, comprising cutting out an inspection area from the captured image and extracting at least the color information from the inspection area thus cut out.

6. An inspection jig for an ophthalmic device to be used in the method for inspecting an ophthalmic device according to claim 1,
the inspection jig comprising the imaging inspection subject.

7. The inspection jig for an ophthalmic device according to claim 6, wherein an optical thin film that reflects illumination light from the illumination optical system is formed at least on a front surface of the imaging inspection subject.

8. The inspection jig for an ophthalmic device according to claim 7, wherein the optical thin film causes specular reflection of the illumination light.

9. The inspection jig for an ophthalmic device according to claim 7, wherein the optical thin film includes a plurality of portions with different thicknesses.

10. The inspection jig for an ophthalmic device according to claim 6, comprising a retaining portion for the imaging inspection subject that retains the imaging inspection subject, and
the retaining portion for the imaging inspection subject is attached to a tip of the objective lens-barrel.

11. An ophthalmic device comprising the inspection jig of an ophthalmic device according to claim 6.
